(19) United States
(12) Patent Application Publication
(10) Pub. No.: US 2008/0288066 A1
(43) Pub. Date: Nov. 20, 2008

(54) Toric sulcus lens

(75) Inventor: J. Stuart Cumming, Laguna Beach, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(73) Assignee: C&C VISION INTERNATIONAL LIMITED

(21) Appl. No.: 11/933,117

(22) Filed: Oct. 31, 2007

Related U.S. Application Data
(60) Provisional application No. 60/938,412, filed on May 16, 2007.

(51) Int. Cl. A61F 2/16

(52) U.S. Cl. 623/6.56

(57) ABSTRACT

There is disclosed herein a “piggyback” cylindrical (toric) intraocular lens for placement in front of an accommodating or standard intraocular lens that is already in the capsular bag of the eye. This additional lens is placed in the sulcus, which leaves a significant space between the two lenses, particularly if the lens in the capsular bag is vaulted backwards.

This application is based on U.S. Provisional application Ser. No. 60/938,412 filed May 156, 2007, which is fully incorporated herein by reference.

### BACKGROUND

The power of intraocular lenses can now, after careful preoperative measurements of the eye's length and the curvature of the cornea, be calculated to give the patient excellent vision providing they do not have astigmatism. Astigmatism is usually caused by the cornea being aspherical, having two different radii.

Various methods have been used to correct the asphericity, called the corneal cylinder. These have been primarily efforts to flatten the cornea by making cuts in the cornea, so-called limbal relaxing incisions. Other surgical techniques have been performed by either intralameller surgical ablation (LASIC) or surface corneal ablation (PRK). Intraocular lenses have also been employed to correct corneal cylinder. The most common form of correction is to place a cylindrical surface on the intraocular lens. These lenses have been either placed in the capsular bag after removing the cortex and nucleus of the human lens, or placed in the posterior chamber on top of the human lens. Contact lenses have also been utilized for many years to correct corneal asphericity.

Recently, accommodating lenses have been developed. These lenses move backward and forward in the eye and the optic deforms when subjected to an increase in vitreous cavity pressure. These accommodating lenses vault posteriorly within the eye and move forward to accommodate; however, their forward movement at its maximum would not allow the optic to touch the toric sulcus-placed non-accommodating lens.

### SUMMARY OF THE INVENTION

An object of this invention is to provide a “piggyback” cylindrical (toric) intraocular lens that can be placed in front of an intraocular lens that is already in the capsular bag. The lens is placed in the sulcus, which leaves a significant space between the two lenses, particularly if the lens in the capsular bag is vaulted backwards.

The implantation can be done during initial surgery of implanted in the eye some time after the primary surgery. The lens is manufactured with several toric powers and no spherical power, and with several toric powers and both positive and negative additional powers. The lens can then be implanted to correct the toric power and/or fine tune the spherical power.

The lens is made from a biocompatible optical material, e.g., silicone, acrylic, hydrogel, or collamer with loops made from either polyimide, prolene, PMMA, or any other biocompatible material.

The lens is either a loop or plate design with either loop or four-point fixation and design such that once placed within the sulcus of the eye it does not rotate or move with accommodation.

An object of the prevention is to provide an improved intraocular lens and lens system.

### BRIEF DESCRIPTION OF THE DRAWINGS

FIG. 1 is a cross-sectional view of the human eye illustrating the lenses of the present invention implanted therein.

FIG. 2a is a plan view of the “piggyback” lens of FIG. 1, and FIG. 2b is a side view thereof.

### DESCRIPTION OF PREFERRED EMBODIMENT

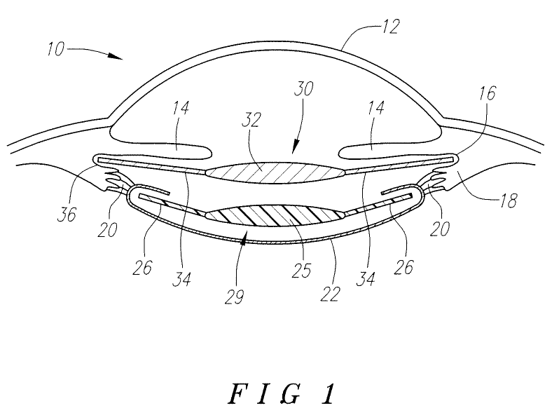

Turning now to the drawings and first to FIG. 1, the same provides cross-sectional view of the human eye 10 showing part of its structure including the cornea 12, iris 14, sulcus 16, ciliary muscle 18, zonules 20, and capsular bag 22. A standard accommodating IOL or standard IOL 24 is shown implanted in the capsular bag and having a lens 25 and haptics 26. This lens 25 can be an accommodating or standard IOL.

The improvement according to the present invention is implantation of the “piggyback” lens as described above which is identified as 30 in FIG. 1 and is a sulcus toric lens having an optic 31, haptics 34 and fixation loops 36 (only the left one being shown) on the ends of the haptics 34.

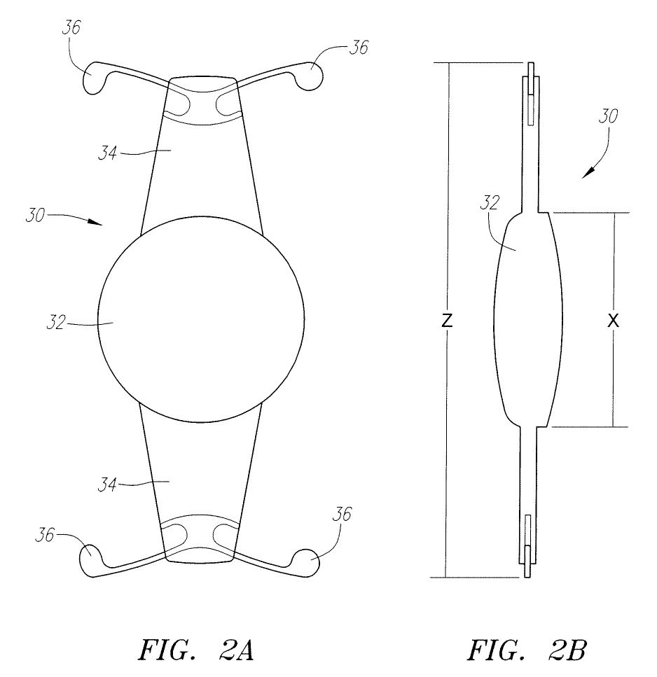

FIGS. 2a and 2b further illustrate the structure of the piggyback lens 30 showing the optic 32, haptics 34 and fixation loops 36 in further detail in FIG. 2a. The lens 30 does not have hinges like accommodating lenses. FIG. 2b is a side view to illustrate the dimension of the diameter of the optic and overall length of the lens 30. The lens 30 can be formed of any optical formable material such as silicone, acrylic, hydrogel or collamer The toric optic 32 preferably has a diameter X of 4.5-7.0 and preferably 6.0 mm with low spherical powers, and various toric powers for each spherical power of 1.00, 1.50, 2.00, 2.50 mm, etc. The loops 36 provide four point fixation and the loops can be of polymide, prolene or PMMA. The distance Y in FIG. 1 can be 11.5-12.5 mm. A typical overall length Z for the toric lens 30 is 10.5-11.5 mm.

While an embodiment of the present invention as been shown and described, various modifications may be made without departing from the scope of the present invention, and all such modifications and equivalents are intended to be covered.

1. An intraocular lens with a toric aspheric surface and haptics for placement into the ciliary sulcus of the human eye in front of a posteriorly placed intraocular lens, a piggy back lens.

2. An intraocular lens according to claim 1 where the haptics both center the lens within the eye and prevent it from rotating.

3. An intraocular lens according to claim 1 where the lens is made from a biocompatible optical material, e.g., silicone, acrylic, PMMA, HEMA or collamer.

4. An intraocular lens according to claim 1 where the haptics and optic are made from a flexible optical material.

5. An intraocular lens according to claim 2 where the haptics are plate haptics.

6. An intraocular lens according to claim 5 wherein the plate haptics have fixation loops on the ends thereof.

7. An intraocular lens according to claim 1 wherein loops are on the ends of the haptics.

8. An intraocular lens according to claim 1 wherein the lens has a toric power and no spherical power.

9. An intraocular lens according to claim 1 where the lens has both a toric power and a spherical power.
